(19) United States
(12) Patent Application Publication
(10) Pub. No.: US 2024/0170116 A1
(43) Pub. Date: May 23, 2024

(54) WELL-BEING RECOMMENDATION SYSTEM

(71) Applicant: Symphony9 LLC, Glenview, IL (US)

(72) Inventors: Santoash Rajaram, Portland, OR (US); Karan Mishra, Glenview, IL (US)

(21) Appl. No.: 17/992,733

(22) Filed: Nov. 22, 2022

(51) Int. Cl. G16H 20/00; G16H 10/20; G16H 50/20; G16H 50/70

(52) U.S. Cl. CPC G16H 20/00; G16H 10/20; G16H 50/20; G16H 50/70

(57) ABSTRACT

Based on the recognition that an individual's well-being is not a function of a single factor or variable but is instead a multi-dimensional function of a number of factors or variables where interventions (i.e., activities that people perform to help with their well-being) affect each individual differently, a system recommends interventions that best influence well-being across multiple factors by creating an n-dimensional profile specific to each individual. Upon completion of the interventions, the profile is refined to develop n-dimensional sub-profiles for each individual. This refining process is informed using the effectiveness of each intervention as it relates to each individual and environmentally specific well-being factors using implicit and explicit signals responsive to the interventions. The recommendation of future well-being interventions is based on well-being factors in the refined n-dimensional well-being profile. The n-dimensional profile enables tracking of the user's well-being journey in n-dimensional space as the user completes interventions.

### TECHNICAL FIELD

This disclosure describes workflows and machine learning models for training a machine learning recommendation model to recommend interventions to advance an individual's well-being and to update one or more well-being profiles that are specific to the user and the user's environment.

### BACKGROUND

A number of consumer wearable products are available that enable users to track various health metrics such as blood pressure, heart rate, activity levels, and the like. Such information is collected by the wearable and is typically presented to the user in a display on the wearable or on a connected mobile device (e.g., a smartphone). Such information is useful to the user to provide a snapshot of the user's condition as it relates to the respective tracked variables; however, the information tracked by conventional wearable products is not sufficient to enable the user to track her well-being over time as the user's well-being is not a function of a single factor or variable and different activities affect different individuals differently.

### SUMMARY

Various examples are now described to introduce a selection of concepts in a simplified form that are further described below in the Detailed Description. This Summary is not intended to identify key or essential features of the claimed subject matter, nor is it intended to be used to limit the scope of the claimed subject matter.

In sample configurations described herein, a method of monitoring a user's well-being is described that includes creating an n-dimensional well-being profile specific to the user, the n-dimensional well-being profile comprising values for n well-being factors, and mapping at least one intervention activity to a plurality of well-being factors impacted by the intervention activity. Then, upon completion of the user performing an intervention activity, the n-dimensional profile is refined into at least one n-dimensional sub-profile for an environment in which the intervention activity was performed, wherein the n-dimensional profile is refined in accordance with implicit and explicit responses to the completed intervention activity that impact the plurality of well-being factors in the environment. A recommendation is provided to the user for another intervention activity based on the refined at least one n-dimensional sub-profile for the environment, and changes in at least one of the n-dimensional well-being profile or the at least one n-dimensional sub-profile are monitored to track changes in the user's well-being over time as the user completes intervention activities. The n well-being factors may include factors that can directly or indirectly affect the user's well-being, including a plurality of cardio, resting heart rate, sleep hours, water intake, or smoking. The method may further include maintaining a library of the n well-being factors in a data store, the n well-being factors including at least one of calm, self-love, forgiveness, modesty, prudence, self-regulation, kindness, love, teamwork, leadership, diet, sleep, drugs, or community. The environment may also include one of work, home, a gym, or driving, where the user has an n-dimensional well-being profile for each environment.

The methods described herein further include assigning weights to well-being factors that are specific to the user and the user's environment to create the n-dimensional well-being profile. In sample configurations, the weights are increased based on a number of implicit and explicit responses triggered by an associated well-being factor in response to an intervention activity.

The methods described herein may further include generating trend and collaborative well-being features. In sample configurations, the trend well-being features extrapolate intervention activities based on several users' completed intervention activities. A specific intervention activity for the environment is recommended based on trends specific to the user. The collaborative well-being features also may include well-being features from other users with a similar well-being profile to the user.

In sample configurations, the implicit and explicit responses relate to physiological and psychological effects of the completed intervention activity impacting the plurality of well-being factors in the environment. The implicit responses may be measured by sensors while the explicit responses may be provided by the user in response to a survey.

In further sample configurations, mapping the at least one intervention activity to the plurality of well-being factors impacted by the intervention activity may include a many-to-many mapping of the implicit and explicit responses to the completed intervention activity to the plurality of well-being factors impacted by the completed intervention activity. Also, an overall well-being profile for the user may be computed based on at least one n-dimensional sub-profile for each environment in which an intervention activity is performed by the user. In sample configurations, the computing may include summing respective well-being factors together for the at least one n-dimensional sub-profile for each environment. In addition, the n-dimensional well-being profile specific to the user may be collaboratively filtered with an n-dimensional well-being profile specific to another user. The resulting recommendation may be provided to the user for another intervention activity based on a result of the collaborative filtering.

The n-dimensional well-being profile specific to the user may be stored as versioned well-being factor values specific to the user and the environment. The n-dimensional well-being profile specific to the user may be stored on a mobile computing device of the user. Also, the n-dimensional well-being profile specific to the user may be encrypted into encrypted data and stored on a server. The n-dimensional well-being profile specific to the user also may be de-identified into de-identified aggregate profile data for a plurality of users and stored on the server.

Also, each time the well-being factors for a given n-dimensional well-being profile are updated, a new version of the n-dimensional well-being profile may be created and a profile table updated to track a version lineage of the n-dimensional well-being profile. In sample configurations, the n-dimensional well-being profile specific to the user may be stored in a hierarchical tree structure on a computing device where each new version of the n-dimensional well-being profile is stored under a current version of the n-dimensional well-being profile to create the at least one n-dimensional sub-profile. In such a configuration, tracking changes in the user's well-being over time as the user completes intervention activities may include tracking the n-dimensional well-being profile specific to the user in the hierarchical tree structure where each node in the tree represents a point in n-dimensional space.

In such configurations, the method may include identifying which well-being factors impact a user's well-being the most at each level in the hierarchical tree structure and recommending intervention activities for the identified well-being factors at each level in the hierarchical tree structure. In such configurations, building the hierarchical tree structure for the user's n-dimensional well-being profile may include the steps of obtaining a list of recommended well-being factors to pursue given the user's current position, recommending intervention activities for each of the listed recommended well-being factors, gathering feedback from the user to obtain an associated well-being label for the recommended well-being factors, creating a new n-dimensional well-being profile for each intervention activity that uses a recommended well-being profile from a previous level in the hierarchical tree structure as its parent, and selecting an n-dimensional well-being profile with a best well-being label as a candidate n-dimensional well-being profile for a current level in the hierarchical tree structure.

In sample configurations, refining the n-dimensional profile into at least one n-dimensional sub-profile for an environment in which the intervention activity was performed may include incrementing values of the at least one n-dimensional sub-profile for the environment by a total number of implicit or explicit responses triggered by the completed intervention activity. The implicit response may be triggered by the completed intervention activity when a value for a well-being factor affected by the completed intervention activity falls outside two standard deviations from historical values associated with the user for the well-being factor.

In further configurations, refining the n-dimensional well-being profile into at least one n-dimensional sub-profile for an environment in which the intervention activity was performed may include determining the user's environment, measuring all implicit and explicit responses associated with well-being factors in the user's environment related to the recommended intervention activity, determining which implicit responses impacted the well-being factors for the user in the user's environment by comparing a value of an implicit response against an historical value for the implicit response in that environment for the user, and when an explicit response is captured, determining when the explicit response was triggered. For each implicit or explicit response that changed in value, the method may further include determining the well-being factors that impact the implicit or explicit response, and for each well-being factor that impacts an implicit or explicit response, fetching records for an environment specific n-dimensional well-being profile specific to the user and determining from the records the values of well-being factors for the user for the environment specific n-dimensional well-being profile specific to the user. A value of at least one well-being factor may be incremented by a count of triggered implicit and explicit responses and the n-dimensional well-being profile updated with the value of the at least one well-being factor.

In still further configurations, a well-being label may be provided as a target variable to optimize while training a recommendation model for use in providing the recommendation to the user for another intervention activity based on the refined at least one n-dimensional sub-profile for the environment. Training the recommendation model for another intervention activity may occur at a periodicity depending on how many intervention activities have been completed. The training may use variables extracted during the completed intervention activity as a feature vector and the well-being label as the target variable for the training. Training the recommendation model for another intervention activity also may use data extracted using a collaborative filter from other users' de-identified aggregate well-being profile data. Training the recommendation model for another intervention activity may further include predicting a sequence of well-being factors that is most likely to result in an improvement to the well-being label based on previous sequences of well-being factors that improved the well-being label in response to intervention activities mapped to the previous sequences of well-being activities.

The description provided herein also relates to a system for monitoring a user's well-being. In sample configurations, such a system includes a hierarchical tree structure that stores an n-dimensional well-being profile specific to a user where each new version of the n-dimensional well-being profile is stored under a current version of the n-dimensional well-being profile to create at least one n-dimensional sub-profile comprising values for n well-being factors, respective well-being factors of the user being mapped to at least one intervention activity that impacts the respective well-being factors of the user. A computing device is further proved to receive implicit and explicit responses to an intervention activity completed by the user that impacts the respective well-being factors of the user in an environment of the user and to refine the n-dimensional well-being profile specific to the user into at least one n-dimensional sub-profile for the environment in which the intervention activity was performed. The n-dimensional well-being profile may be refined in accordance with the implicit and explicit responses to the completed intervention activity. The computing device may also monitor changes in at least one of the n-dimensional well-being profile or the at least one n-dimensional sub-profile to track changes in the user's well-being over time as the user completes intervention activities. A personalized recommendation model may be further included to provide a recommendation to the user for another intervention activity based on the refined at least one n-dimensional sub-profile for the environment.

The system may further include a server that receives and stores encrypted intervention activity completion data from the computing device and processes the intervention activity completion data to form a de-identified data stream. The server may further process the de-identified data stream for a plurality of users to generate intervention activity recommendations for the user and pushes the intervention activity recommendations to the computing device. A personalized training module may be used to train the personalized recommendation model using data extracted from the implicit and explicit responses to the completed intervention activity.

A method may be performed by the apparatus, and further features of the method result from the functionality of the apparatus. Also, the explanations provided for each aspect and its implementation apply equally to the other aspects and the corresponding implementations. The different configurations may be implemented in hardware, software, or any combination thereof. Also, any one of the foregoing examples may be combined with any one or more of the other foregoing examples to create a new configuration within the scope of the present disclosure.

### DETAILED DESCRIPTION OF THE DRAWINGS

The foregoing and other beneficial features and advantages of the subject matter described herein will become apparent from the following detailed description in connection with the attached figures, of which:

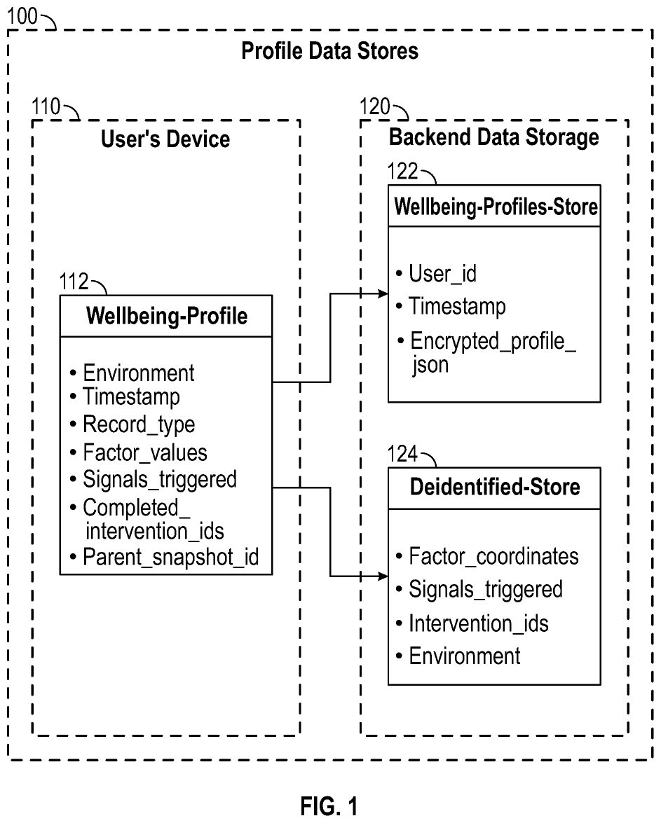

FIG. 1 illustrates a diagram of profile data stores for storing well-being profile data of a user in a sample configuration.

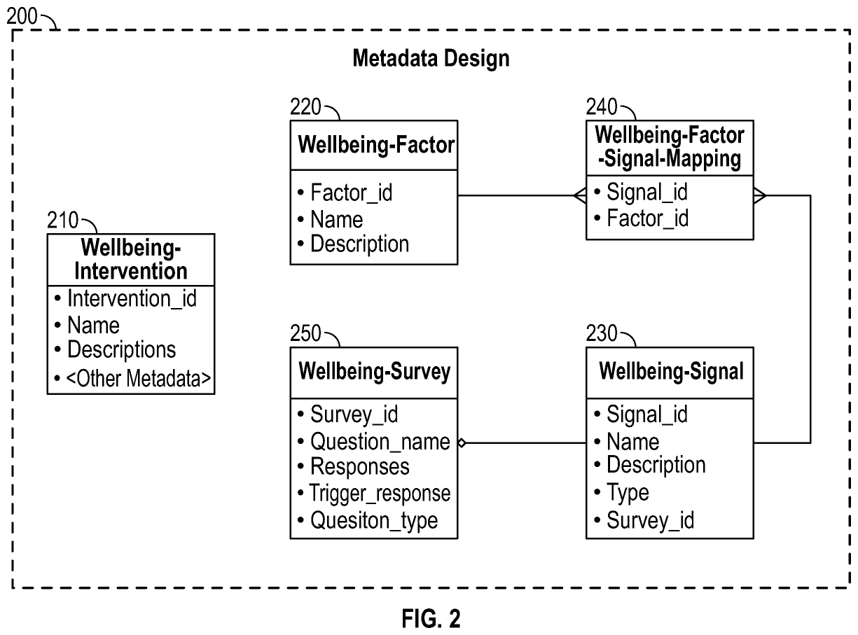

FIG. 2 illustrates a diagram of a high-level metadata model of explicit signals in a sample configuration.

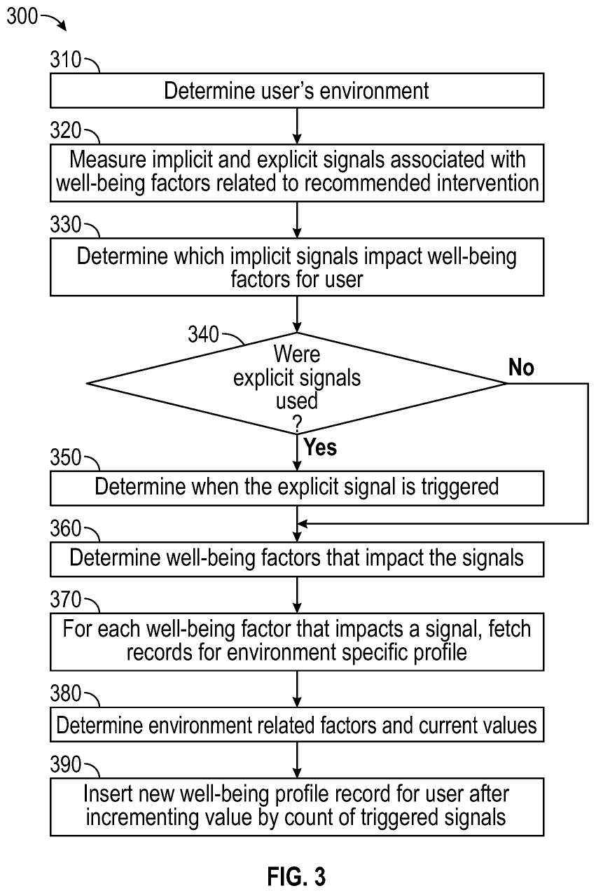

FIG. 3 is a flow chart illustrating a well-being profile update routine when the user completes an intervention in a sample configuration.

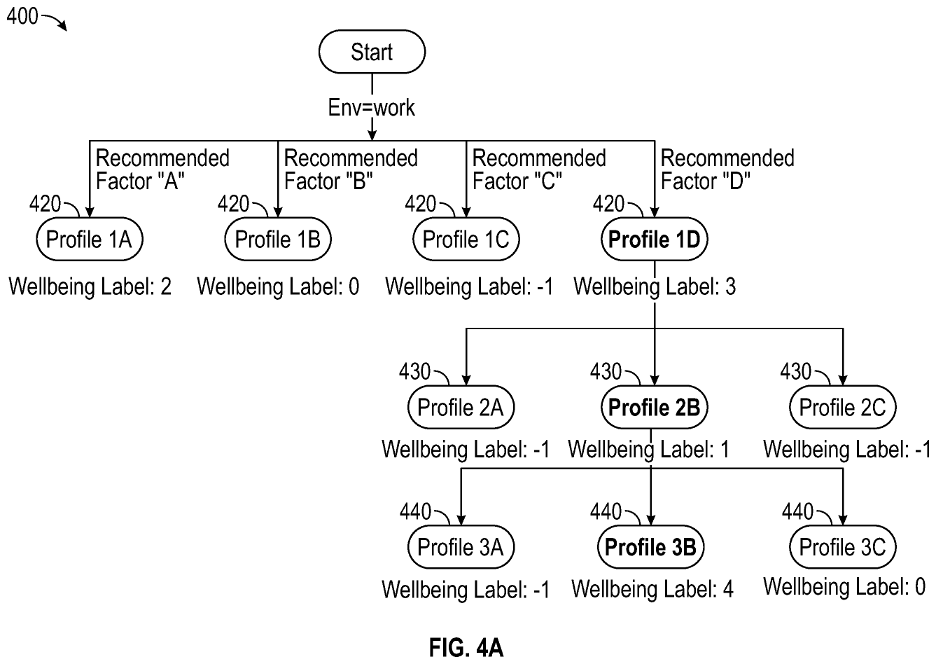

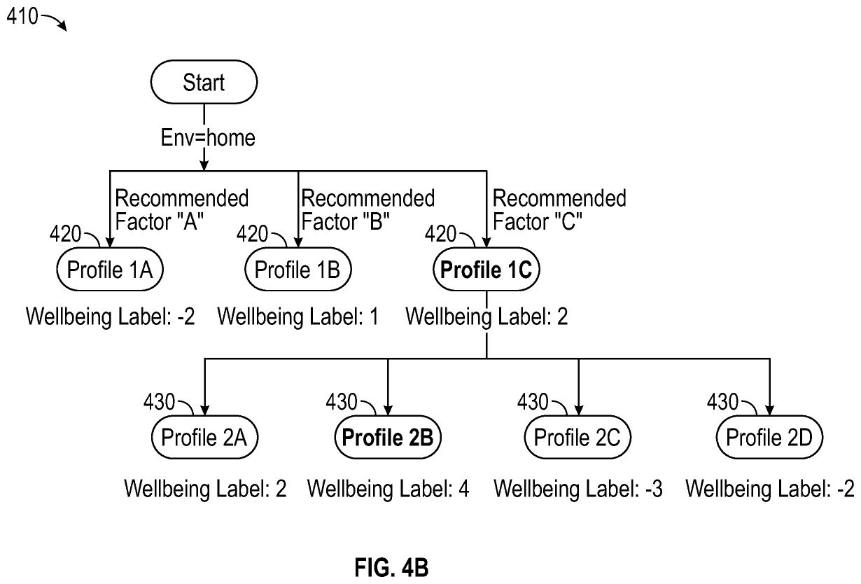

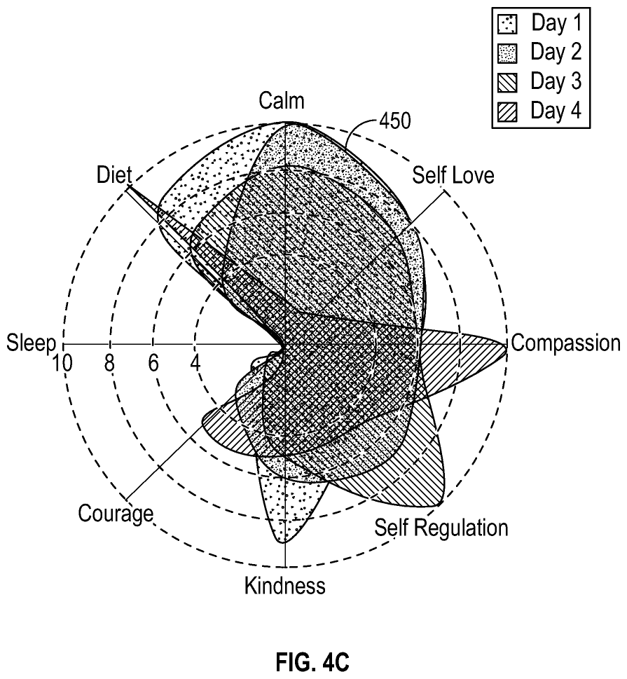

FIG. 4A is a diagram illustrating a hierarchical data structure for a work environment in a sample configuration.

FIG. 4B is a diagram illustrating a hierarchical data structure for a home environment in a sample configuration.

FIG. 4C is a diagram illustrating a user's well-being tracked over several days in n-dimensional space for well-being factors in a sample configuration.

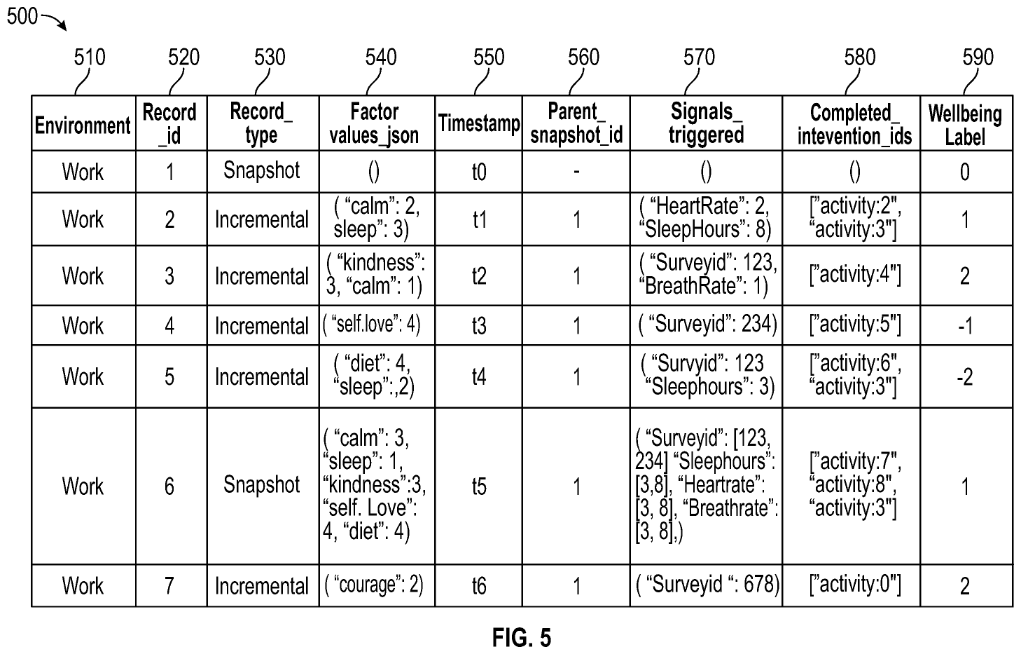

FIG. 5 is a table illustrating a database model for storing well-being profile data on a mobile device in a sample configuration.

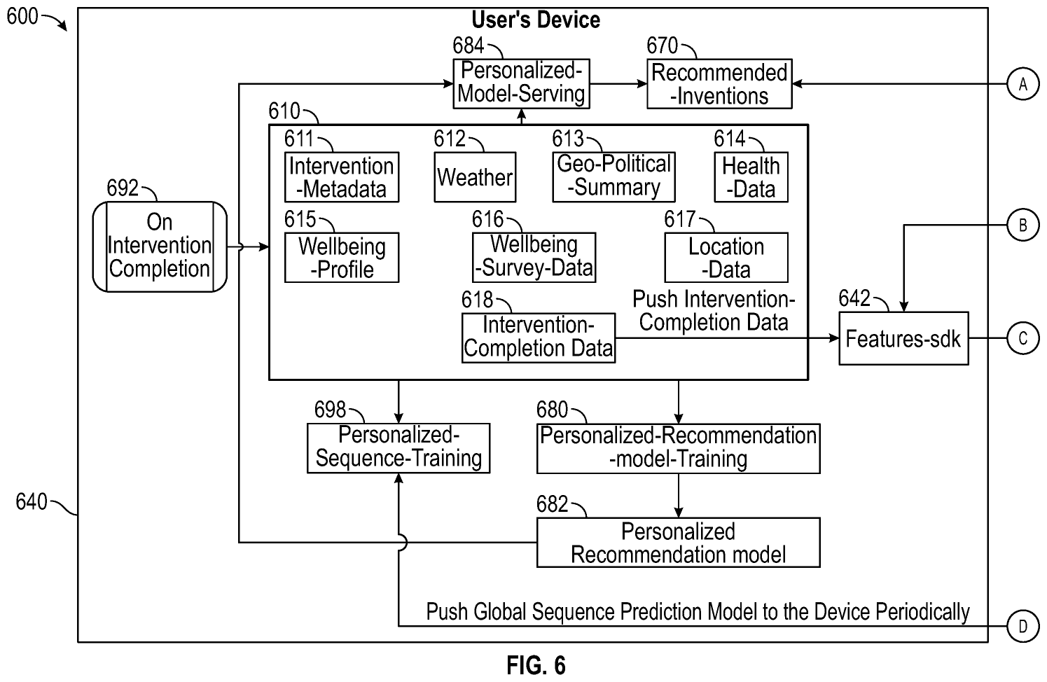

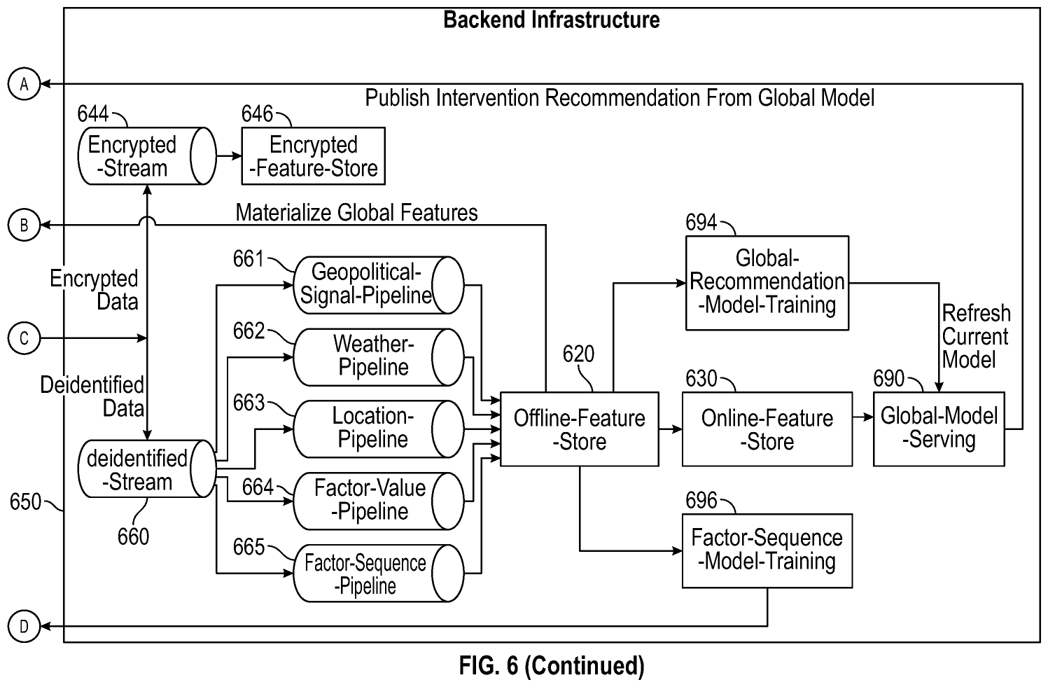

FIG. 6 is a diagram of a high-level view of components of the recommendation infrastructure in a sample configuration.

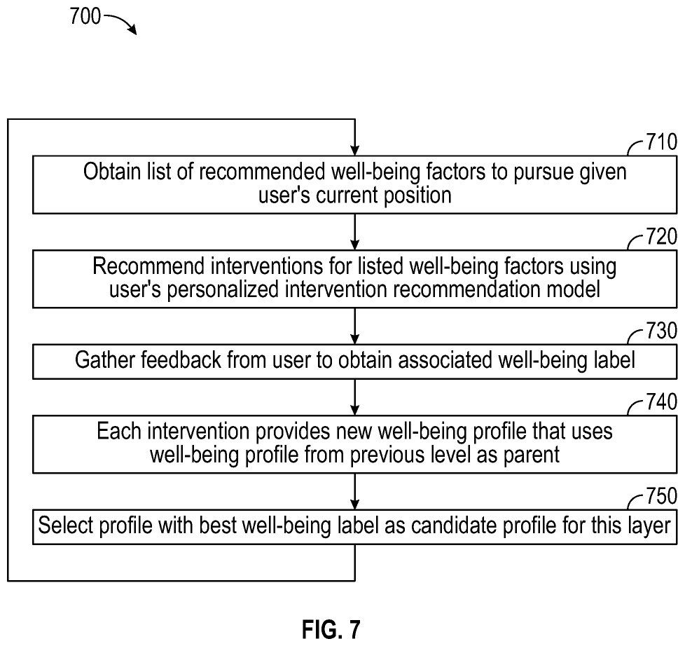

FIG. 7 is a flow chart illustrating the building of a well-being profile hierarchy in a sample configuration.

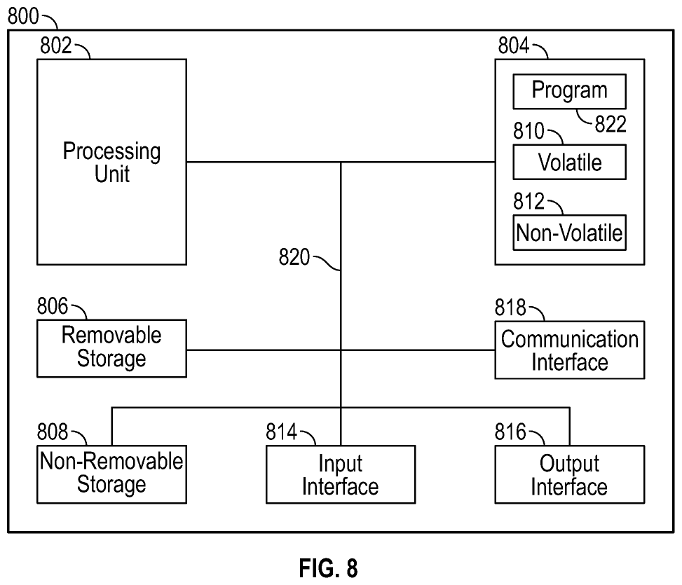

FIG. 8 is a block diagram illustrating circuitry for performing methods and implementing processing features according to the sample configurations.

### DETAILED DESCRIPTION OF ILLUSTRATIVE CONFIGURATIONS

Sample configurations will be described with respect to FIGS. 1-8 for use in monitoring the well-being of a user, although those skilled in the art will appreciate that the teachings provided herein may be used for other purposes such as tracking the well-being of an animal or a group of individuals based on observable data that may be captured using conventional techniques. Such configurations are intended to be within the scope of the present disclosure.

The present disclosure is based on the recognition that an individual's well-being is not a function of a single factor or variable but is instead a multi-dimensional function of a number of factors or variables where intervention activities (i.e., activities that people perform to help with their well-being; hereinafter “interventions”) affect each individual differently. The system described herein recommends interventions that best influence well-being across multiple factors by creating an n-dimensional profile specific to each individual. Upon completion of the interventions, that profile is refined to develop n-dimensional sub-profiles for each individual. This refining process is informed using the effectiveness of each intervention as it relates to each individual and environmentally specific well-being factors using implicit and explicit responses (herein referred to as “signals”) responsive to the interventions. The recommendation of future well-being interventions is based on well-being factors in the refined n-dimensional well-being profile. The n-dimensional profile enables tracking of the user's well-being journey in n-dimensional space as the user completes interventions.

As used herein, well-being factors are factors that can directly or indirectly affect an individual's well-being, like cardio, resting heart rate, sleep hours, water intake, smoking, etc. Well-being environments for an individual reference the specific circumstances under which an individual would carry out the particular intervention. The process of assigning weight to a well-being factor is specific to a user and the user's environment and the resultant mapping is called a well-being profile. The system described herein aims to map as many factors as possible to respective dimensions for a given user in a specific environment. Each user can have multiple well-being profiles associated with them, one for each environment. These profiles drive the recommendation of interventions for a user.

In sample configurations, three kinds of data are used to power the recommendation of interventions for a user: user data, intervention data, and user environment data. User data includes all the data that is specific to a user, including age, gender, demographics, health data, nutrition data, sleep data, intervention completed data, calendar data, survey response data, etc. Intervention data refers to the metadata about a given intervention, including name, description, associated morbidity factors, location, suitable environment, etc. User environment data refers to the data about the user's current environment. The user environment data determines a user's current physical environment or situation, like work, home, a gym, driving, etc.

Once the user data, intervention data, and user environment data has been collected, trend and collaborative features are generated to feed into the recommendation engine. Trend features extrapolate relevant interventions based on several users' completed interventions and recommend a specific intervention based on the trends specific to the user. These interventions are specific to the environment in which these interventions are being performed. Trends will help recommend appropriate interventions for the given location, like volunteering opportunities, fitness classes, etc. Collaborative features extract features from other users with a similar profile. This data could include interests, tags, dimensions, etc.

User and user environment data are collected when an intervention is completed. Based on the data collected, an application measures the effectiveness of the intervention. Effectiveness is measured based on the response to the survey at the time of intervention completion and the physiological (using wearable and other measurable data like heart rate, sleep hours, etc.) and psychological effects (using survey based explicit signals) that are determined as a result of the completed intervention. The well-being profile is amended to reflect the updated physiological and psychological effects. Since a user can have multiple well-being profiles depending on the environment, the appropriate well-being profile will be altered based on the environment in which the user performed the intervention.

The overall well-being profile of an individual is computed based on the environment-specific well-being profiles. This overall wellbeing profile is calculated by summing the individual factor values together to form the computed well-being which places the user in an n-dimensional space. This way of representing well-being allows the system to track the user's well-being journey in n-dimensional space as the user completes interventions. In sample configurations, the system further may use collaborative filtering techniques to figure out similar journeys that have been successful in the past to recommend new well-being factors and interventions to the user.

### Wellbeing Profile

As used herein, a wellbeing profile stores the versioned factor values specific to a user and an environment. The user's wellbeing profile is stored and maintained for the user in a user computing device such as a mobile phone. The techniques for building a well-being profile for a user are described further below. The data model backing the well-being profile is described first.

In sample configurations, the well-being profile of a user stores the user data, intervention data, and environment data using the design shown in FIG. 1. As illustrated in FIG. 1, there are two components to the profile data stores 100, an on-device store 110 and a backend data store 120, which is typically maintained on a remote server. The on-device store 110 contains user-specific profile data 112 such as environment data, factor values, intervention data, and the like. On the other hand, the backend data store 120 is used to track user-specific encrypted data 122 (e.g., user ID, timestamps, and encrypted profile data) and de-identified aggregated profile data 124 (e.g., well-being factor coordinates, signals triggered, intervention IDs, and environment data) to help with recommendations.

The well-being factors are made up of a number of factors that are granular abstractions that contribute toward well-being. For example, well-being factors may include calm, self-love, forgiveness, modesty, prudence, self-regulation, kindness, love, teamwork, leadership, diet, sleep, drugs, community, etc. In sample configurations, a library of well-being factors may be maintained in a datastore with metadata.

All measures of well-being, implicit and explicit, also may be maintained in the profile data stores 100. Implicit signals are signals that are measured (e.g., by sensors) without any initiative from the user, whereas explicit signals are usually a form of a survey. Metadata about the implicit signals may include name, description, device (sensor) that measures it, expected range, etc. Metadata about explicit signals may consist of the survey and how often the survey data is captured. A mapping of the implicit and explicit signals to well-being factors may be maintained so that a determination can be made as to what well-being factors are affected at the time of the intervention completion. It will be appreciated that this mapping is a many-to-many relationship. In other words, the same implicit or explicit signal can contribute to several well-being factors, and each well-being factor can be affected by several implicit and/or explicit signals. This mapping may be maintained in accordance with the latest research regarding a person's well-being.

Interventions track the task to be performed by the user to improve her well-being. The interventions are recommended to the user to address a specific well-being factor(s) in a given environment. Once the user completes the intervention, implicit and/or explicit signals associated with the well-being factors (that the intervention was recommended for) will be measured or captured. The well-being factor value in the user's well-being profile(s) will be incremented by the total number of implicit or explicit signals triggered. For example, in order for an implicit signal to be triggered, it may be required to fall outside two standard deviations from the historical values associated with that specific user. The explicit signals, on the other hand, may contain metadata as illustrated by way of example in FIG. 2 to indicate how the specific intervention affects that wellbeing factor(s).

FIG. 2 illustrates a diagram of a high-level metadata model 200 of explicit signals in a sample configuration. As illustrated, the metadata model includes well-being intervention data 210, well-being factors 220 that are mapped to well-being signals 230 via well-being signal mapping 240, and well-being survey data 250.

In sample configurations, the well-being system described herein triggers a well-being profile update routine 300 when the user completes an intervention. A sample configuration of the well-being profile update routine 300 is shown in FIG. 3.

At 310, the user's environment is determined. The environment could be input by the user or may be determined automatically based on the user's location, user behavior, etc.

At 320, all the implicit and explicit signals associated with well-being factors in the user's environment related to the recommended intervention are measured. If surveys are needed to capture the explicit signals, the user will be asked relevant questions in the survey.

Once the implicit signals have been measured, a determination is made at 330 to determine which implicit signals impacted the well-being factors for the specific user in the user's environment by comparing the value of the implicit signal against the user's historical value for the implicit signal in that environment.

A determination is made at 340 as to whether explicit signals (surveys) were used to capture user intervention response data. If so, the metadata in the survey will be used at 350 to determine when the explicit signal is triggered (e.g.: Answering “yes” to a question may be considered a trigger.)

For each implicit or explicit signal that changed, a determination is made at 360 to determine the well-being factors that impact the implicit or explicit signals.

For each well-being factor that impacts a specific signal, records are fetched at 370 for the environment specific profile, and related well-being factors for the user and the current values are determined at 380. If no matching records were found, the current value is assumed to be zero.

At 390, a new well-being profile record for the user is inserted after incrementing the value by the count of triggered signals.

It is noted that the well-being profile is a versioned entity, so any change made to the well-being profile is added as a new row in the data store. This versioning system allows the system to track a user's well-being journey over time. Each time the factors are updated for a given profile, a new version of the profile is created. The parent id column in the profile table is used to keep track of the lineage.

A well-being label may be provided as an explicit input from the user regarding the user's well-being upon intervention completion. The well-being label may be measured using different questions. The responses to these questions may be normalized to a value between−2 to 2 (ranging from stressed to invigorated). Of course, other ranges may be used for the well-being label, as appropriate. This well-being label then may be used as a target variable to optimize while training a recommendation model for the user.

### N-Dimensional Space

In sample configurations, each well-being profile is stored in a hierarchical data structure on the user's mobile device (e.g., smartphone). For example, FIG. 4A is a diagram illustrating a hierarchical data structure 400 for a work environment, while FIG. 4B is a diagram illustrating a hierarchical data structure 410 for a home environment. As illustrated, hierarchical data structures 400 and 410 include respective well-being profiles 420 for each recommended well-being factor for a corresponding well-being label. As also illustrated, the respective well-being profiles 420 may, in turn, include one or more additional well-being profiles 430 in the same branch that relate back to a corresponding parent well-being profile 420. Similarly, the respective well-being profiles 430 may include one or more additional well-being profiles 440 (FIG. 4A) in the same branch that relate back to corresponding parent well-being profiles 420 and 430 based on the recommended well-being factors, and so forth.

As illustrated in FIGS. 4A and 4B, each new version of the user's well-being profile is stored under the current version to create a sub profile. In order to fetch the latest version of a well-being profile for a given environment, the last node in the tree is traversed. This allows the system to maintain an environment specific lineage of the journey. The latest profiles from individual journeys are combined to determine the overall well-being profile of the user. Similar to environment specific well-being profiles, overall well-being profiles also may be versioned and tracked for each user.

If each well-being factor is considered as a plane 450 in a n-dimensional space as shown in FIG. 4C, the current position of the user may be represented in that n-dimensional space. As shown in FIG. 4, the user's well-being is tracked over several days in n-dimensional space for well-being factors including calm, self-love, compassion, self-regulation, kindness, courage, sleep, and diet, for example. Since the user's well-being journey is tracked by the well-being factors, the user's wellbeing journey also may be tracked over time in that n-dimensional space. Also, by representing the user's well-being journey in the n-dimensional space, user interventions may be recommended that work holistically across several well-being factors and the results of the interventions may be readily tracked. Also, tracking the user's well-being journey in n-dimensional space allows successful journeys from other users to be found and used to recommend further interventions. For example, profiles that are closer together on the well-being journey in n-dimensional space can be influenced by the similar interventions for the same or a different user.

### Well-being Profile Storage

The well-being profile is specific to an individual, so it needs to be tracked privately on the user's mobile or other computing device. To support multiple computing devices for a given user, the profile data may be stored in a backend data storage (e.g. backend data storage 120 in FIG. 1) encrypted with a user-specific encryption key. This database is treated as long-term permanent storage of the well-being profile data, whereas the on-device storage is treated as a read-through and write-through cache. The system may publish updates to individual computing devices when the user's well-being profile data is updated in the backend data storage to help keep the computing device's well-being profile information updated.

FIG. 5 is a table illustrating a database model for storing well-being profile data on a mobile device in a sample configuration. As illustrated, the database model may be represented as a table 500 that stores environment data 510 for the environment from which the data has been collected, a record identifier 520, a record type 530 (e.g., snapshot or incremental data), well-being factor values (e.g., in JavaScript Object Notation (JSON) format) 540, a timestamp 550, a parent snapshot identification 560 (e.g., relates snapshot data to parent snapshot data), signals triggered data 570, completed intervention IDs 580, and well-being label 590 (in this example on a scale from −2 to +2). Other data may be stored in the database model for particular applications.

In sample configurations, as users complete the interventions, the well-being factors that are affected are calculated via captured implicit and explicit signals. Weights are assigned to the well-being factors based on the logic described above with respect to FIG. 3. For example, the weight of a well-being factor is increased by the number of signals that were “triggered” (as determined at step 350 in FIG. 3). For instance, if there are two signals (heart rate and number of breaths per minute) related to the well-being factor “calm” that were triggered at the end of an intervention, there will be an increase in the weight of calm by 2.

As shown in FIG. 5, the database model includes two types of records: incremental and snapshots. Incremental records keep track of the incremental changes to the factor values, whereas the snapshots roll up all incremental factor values that have been accumulated since the last snapshot. Each of these updates is inserted into the on device data store as it is calculated. Each record keeps track of the parent snapshot via parent_snapshot_id. If the current record is an incremental update, the parent_snapshot_id column keeps track of the last profile snapshot that it is mutating. If the current record is a snapshot, then it points to its parent profile. To figure out the user's well-being journey in this environment, the incremental records can be ignored. Instead, the last snapshot is found and the parent_snapshot_Cid column is followed to traverse the lineage. Snapshot records are inserted by a scheduling command (e.g., cron job) every predetermined number of hours. Non-environment specific, overall profiles may be calculated along with the snapshots. The overall models are modeled and tracked just like any environment specific models. The snapshot records in the on-device data store 110 may keep track of the user's well-being coordinates in n-dimensional space to track the user's wellbeing progress.

Since the well-being profile data is specific to individual users, it cannot be shared with other users without an explicit approval from the user. In order to make it convenient for users to migrate between computing devices, the users' well-being profile data is stored in the backend data store 120. This data will be encrypted using the key created by the user so this data is opaque to the system. The encrypted storage in the backend data store 120 tracks snapshot data for each user by timestamp. Any update to a given user's profile will be published to all the user's computing devices to keep the well-being profile data store on the user's computing devices up to date. In addition to the user's encrypted profile data, de-identified profile data may be stored in a generic store on the backend data store 120 which tracks the coordinates of the n-dimensional space, the activity IDs that triggered any change, and the signals corresponding to the activity IDs. This de-identified data may be used to add collaborative recommendations based on what worked for other users.

### Recommendations

The system recommends interventions that have the most impact on a user in an environment on a given day. In order to train a model that has the most impact on a given user, the recommendation may be a result of combining results from a personalized recommendation model and global recommendation model.

### Personalized Intervention Recommendation Model

The personalized intervention recommendation model is trained using the following broad categories of data collected specifically for a user:

Intervention data including tags, keywords, etc.
Environment where the intervention was performed
User's health data during the intervention completion
User's location when performing the intervention
Survey responses collected from the user to evaluate the user's well-being
Weather conditions during intervention completion
Other user's nearby when performing the intervention
Geopolitical signals on that day
Factors and values from the wellbeing-profiles as a result of intervention completion
Well-being Label (target variable)

The personalized intervention recommendation model is trained periodically depending on how many interventions were completed. Model training uses all the variables extracted during intervention completion as the feature vector and the well-being label as the target variable. At a high level, the feature vector includes 4 kinds of data:
1. user (health, well-being factor values)
2. environment (location, weather, geopolitical signals, etc.)
3. intervention data
4. other users nearby
   There are several pre-trained deep learning models that may be trained and used in sample configurations. Sample pre-trained deep learning models that may be used to train the personalized intervention recommendation model include DeepFM, Collaborative Deep Learning, and Hybrid Deep Learning algorithms.

Once the model is trained using the feature vectors and the well-being label, the recommendation may be dynamically based on where the user is and what is happening around the user at a given time. The input to the model to get a recommended list of activities may be as follows:
Environment
User's health profile (stressed, sedentary, high movement, etc.)
User's location
Well-being factors to be addressed
Geopolitical profile (regular, good, bad, etc.)
Other users involved

Thus, the input to the personalized intervention recommendation model is mostly based on the user's current surroundings and health data except for the well-being factors to be addressed. For the personalized intervention recommendation model, the well-being factor to be addressed is derived based on what worked for the user in the past under the given conditions (where such data is available; otherwise the model may be assigned a starting value that is adjusted by responses to interventions). This personalized invention recommendation model uses the data available on the user's computing device to train and recommend interventions. Accordingly, the interventions recommended using the personalized intervention recommendation model will not have any variations. In order to discover new interventions, interventions may be recommended based on what other users are doing.

### Global Intervention Recommendation Model

The global intervention recommendation model may be trained using the de-identified data from all the users of the system. If a user is willing to share their intervention and health data anonymously, then the following factors are sent to the backend storage for training:
Intervention data including tags, keywords, etc.
Environment where the intervention was performed
Location where the intervention was performed
Weather conditions during intervention completion
Geopolitical signals
Factors affected
   The input to the global intervention recommendation model may include, inter alia:
Environment
Location
Geopolitical profile (regular, good, bad, etc.)
Well-being factors to be addressed

In the case of the global intervention recommendation model, the well-being factors to be addressed may be derived using a combination of:
1. What well-being factors are users around them working on?
2. What well-being factors are trending on a given day across the globe?
3. What well-being factors are least represented in the user's profile? The first two signals may be extracted using a collaborative filter from other users' de-identified intervention completion data. This logic is designed to introduce new factors that are more relevant in the current environment, thereby introducing variability.

### Well-being Factor Sequence Prediction

In order to figure out which well-being factors to pursue at any given point in time, the system may predict a well-being factors sequence that is mostly likely to result in a better well-being label. This prediction may be treated as a sequence prediction problem. There are several sequence prediction deep learning models available including the most popular one by Google called seq2seq. In order to train the deep learning model, the sequence of factors in the individual's well-being journey across all the users is used to train the global intervention recommendation model. The global intervention recommendation model is fine-tuned on the computing device by using the well-being factors sequence from the last 90 days. The training data for the sequence prediction contains the previous list of well-being factors explored as the input and the well-being factor that worked as the well-being label. Sample training data may include, for example, the training data of the type shown in Table 1 below.

TABLE 1

| | |
|---|---|
| Factor Sequence | Next Item |
| Love Kindness | Calm |
| Smile Transcend Sleep | Stretch |
| Creativity Play Bravery | Forgiveness |

### Recommendation Infrastructure

FIG. 6 is a diagram of a high-level view of components of the recommendation infrastructure 600 in a sample configuration. As illustrated in FIG. 6, there are three types of well-being features stores involved in this machine learning infrastructure: an on-device feature store 610 that stores personalized features, an off-line feature store 620 that stores global data, and an on-line feature store 630 that stores global data. As shown, the personalized features may include intervention metadata 611, weather data 612, geopolitical data 613, user health data 614, the user's well-being profile 615, the user's well-being survey data 616, location data 617, intervention completion data 618, and the like. The user's computing device (e.g., smartphone) 640 pushes an update to the backend infrastructure (e.g., server) 650 whenever there is a change in the environment. For example, the intervention completion data may be pushed to a features software development kit (SDK) 642 that encrypts the data into an encrypted data stream 644 that is provided to the encrypted feature store 646. The features SDK 642 also processes the intervention completion data to de-identify the data to form de-identified data stream 660. The backend infrastructure 650 will push intervention recommendations 670 to the user's computing device 640 when intervention recommendations are available. The recommended interventions 670 are queued to the user's device 640 as and when they become available.

On-device personalized recommendation model training module 680 uses the data that is stored in the on-device feature store 610 to train the personalized recommendation model 682. As noted above, the personalized recommendation model 682 may be a pre-trained deep learning model such as DeepFM, Collaborative Deep Learning, and Hybrid Deep Learning algorithms. If the performance of the new personalized recommendation model 682 is better than the existing personalized recommendation model, then the on-device personalized model serving module 684 will be updated to use the new personalized recommendation model.

The global intervention recommendation models depend on the de-identified data stream 660 and the well-being features (e.g., geopolitical signals 661, weather 662, location 663, well-being factor values 664, factor sequences 665, etc.) extracted from the de-identified data stream 660. The well-being features extracted from the de-identified data stream 660 are stored in the off-line feature store 620. These off-line features will be materialized into the online feature store 630, which in turn is used by the global model server 690 to recommend interventions 670. Global well-being features from the off-line features stored in the off-line feature store 620 are also materialized (condensed) into well-being features that are provided to the features SDK 642 and the on-line feature store 630.

The encrypted feature store 646 is used only as a backup when the user loses the data in the user's computing device 640.

During system operation, intervention completion data is provided at 692 to the on-device feature store 610 in response to sensor measurements, survey inputs, and the like. The invention completion data 618 is stored in the on-device feature store 610 and pushed to the features SDK 642 that encrypts the data into the encrypted data stream 644 that is provided to the encrypted feature store 646. The de-identified data stream 660 created by the features SDK 642 is stored in the off-line feature store 620 and used for the global intervention recommendation training at 694. The updated global recommendation model is provided to the global model server 690. In addition, the de-identified data stored in the off-line feature store 620 is used for factor sequence model training at 696. The updated factor sequence model is used in conjunction with personalized sequence training at 698 on the user's device 640.

### Building Well-being Profile Hierarchy

As noted above, the users' well-being profiles may be tracked in a hierarchical tree structure where each node in the tree represents a point in the n-dimensional space. To proceed from one level to the next, the system identifies which of the planes (well-being factors) impact the user's well-being the most. In other words, at each level in the hierarchy, the plane(s) are chosen on which to move the user. Interventions are recommended for those planes and user feedback is monitored to select the planes to pursue.

For example, if the intervention recommendation resulted in a better well-being label, the resulting well-being profile may be chosen as the candidate for this level and the system proceeds to experiment with the next level. Conceptually, this results in a well-being journey that has the most impact on the user's well-being. Tactically, this approach allows the system to collect the data needed to define the well-being fingerprint of the user.

FIG. 7 is a flow chart 700 illustrating the building of a well-being profile hierarchy in a sample configuration.

At 710, the system obtains a list of recommended well-being factors to pursue given the user's current position. This recommendation may be based on personal history and collaborative filtering across multiple users.

At 720, the system recommends interventions for each of the listed well-being factors using the user's personalized intervention recommendation model.

At 730, the system gathers feedback from the user to obtain the associated well-being label.

At 740, each of the interventions results in a new well-being profile that uses the selected well-being profile from the previous level as its parent.

At 750, the profile with the best well-being label is selected as the candidate profile for this layer and steps 710-750 are repeated.

Thus, unlike systems that may track a user's well-being based on a change in a measured variable, the system described herein represents the user's well-being data in n-dimensions, which enables the user's well-being to be tracked over time and compared to the well-being of other similarly situated users. The training of the recommendation models enables the intervention recommendations to be regularly updated to provide personalized recommendations to the user that will have a maximum impact on the user's well-being profile. The relationships between the well-being factors and the well-being signals responsive to interventions may be updated based on the collected data as well as the latest medical data from experts in the field, thus providing constant improvement in the system's ability to monitor the user's well-being. Feedback on a display of the user's computing device (e.g., mobile phone) enables the user to monitor her multi-dimensional well-being profile and to observe the specific impacts the interventions have on her well-being over time.

### Sample Computer Platforms for Implementing Well-being System

FIG. 8 illustrates one example of a computing device in the form of a computer 800 that may include a processing unit 802, memory 804, removable storage 806, and non-removable storage 808 for implementing the processing features described above in a user client device and/or a server. Although the example computing device is illustrated and described as computer 800, the computing device may be in different forms in different configurations. For example, the computing device may comprise the user's computing device as well as the backend infrastructure described herein. The computing device may be a smartphone, a tablet, smartwatch, or other computing device including the same or similar elements as illustrated and described with regard to FIG. 8. Devices, such as smartphones, tablets, and smartwatches, are generally collectively referred to as mobile devices or user equipment. Further, although the various data storage elements are illustrated as part of the computer 800, the storage may also or alternatively include cloud-based storage accessible via a network, such as the Internet or server-based storage.

Memory 804 may include volatile memory 810 and non-volatile memory 812. Computer 800 also may include, or have access to a computing environment that includes, a variety of computer-readable media, such as volatile memory 810 and non-volatile memory 812, removable storage 806 and non-removable storage 808. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 800 may further include or have access to a computing environment that includes input interface 814, output interface 816, and a communication interface 818. Output interface 816 may include a display device, such as a touchscreen, that also may serve as an input device. The input interface 814 may include one or more of a touchscreen, touchpad, mouse, keyboard, camera, one or more device-specific buttons, one or more sensors integrated within or coupled via wired or wireless data connections to the computer 800, and other input devices.

The computer 800 may operate in a networked environment using communication interface 818 to connect to one or more remote computers. The remote computer may include a personal computer (PC), server, router, network PC, a peer device or other common network switch, or the like. The communication connection accessed via communication interface 818 may include a Local Area Network (LAN), a Wide Area Network (WAN), cellular, Wi-Fi, Bluetooth, Zigbee, or other networks. According to one configuration, the various components of computer 800 are connected with a system bus 820.

Computer-readable instructions stored on a computer-readable medium are executable by the processing unit 802 of the computer 800, such as a program 822. The program 822 in some configurations comprises software that, when executed by the processing unit 802, performs operations according to any of the configurations and models included herein. A hard drive, CD-ROM, and RAM are some examples of articles including a non-transitory computer-readable medium, such as a storage device. The terms computer-readable medium and storage device do not include carrier waves to the extent carrier waves are deemed too transitory. Storage can also include networked storage, such as a storage area network (SAN). Computer program 822 may be used to cause processing unit 802 to perform one or more methods or functions described herein.

It should be further understood that software including one or more computer-executable instructions that facilitate processing and operations as described above with reference to any one or all of steps of the disclosure may be provided in user portable electronic client devices to implement the features described herein. Alternatively, the software may be obtained and loaded into a server for implementing the features described herein through interaction with a user's client device. The software may be stored on a server for distribution over the Internet, for example.

Also, it will be understood by one skilled in the art that this disclosure is not limited in its application to the details of construction and the arrangement of components set forth in the above description or illustrated in the drawings. The configurations herein are capable of other configurations, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The components of the illustrative devices, systems and methods employed in accordance with the illustrated configurations may be implemented, at least in part, in digital electronic circuitry, analog electronic circuitry, or in computer hardware, firmware, software, or in combinations of them. These components also may be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program may be written in any form of programming language, including compiled or interpreted languages, and it may be deployed in any form, including as a stand-alone program or as a module, component, subroutine, or other unit suitable for use in a computing environment. A computer program may be deployed to be executed on one computer or on multiple computers at one site or distributed across multiple sites and interconnected by a communication network. Also, functional programs, codes, and code segments for accomplishing the systems and methods described herein may be easily construed as within the scope of the disclosure by programmers skilled in the art to which the present disclosure pertains. Method steps associated with the illustrative configurations may be performed by one or more programmable processors executing a computer program, code or instructions to perform functions (e.g., by operating on input data and generating an output). Method steps may also be performed by, and apparatus may be implemented as, special purpose logic circuitry, e.g., an FPGA (field programmable gate array) or an ASIC, for example.

The various illustrative logical blocks, modules, and circuits described in connection with the configurations disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. As used herein, a “plurality” comprises two or more.

Processors suitable for the execution of a computer program include, by way of example, both general and special purpose microprocessors, and any one or more processors of any kind of digital computer. Generally, a processor will receive instructions and data from a read-only memory or a random-access memory or both. The elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, magneto-optical disks, compact disc ROM (CD-ROM), or digital versatile disc ROM (DVD-ROM). The processor and the memory may be supplemented by, or incorporated in, special purpose logic circuitry.

Those of skill in the art understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those skilled in the art may further appreciate that the various illustrative logical blocks, modules, circuits, and algorithm steps described in connection with the configurations disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. A software module may reside in random access memory (RAM), flash memory, ROM, EPROM, EEPROM, registers, hard disk, a removable disk, a CD-ROM, or any other form of storage medium known in the art. A sample storage medium is coupled to the processor such the processor may read information from, and write information to, the storage medium. In the alternative, the storage medium may be integral to the processor. In other words, the processor and the storage medium may reside in an integrated circuit or be implemented as discrete components.

As used herein, “machine-readable medium” means a device able to store instructions and data temporarily or permanently and may include, but is not limited to, random-access memory (RAM), read-only memory (ROM), buffer memory, flash memory, optical media, magnetic media, cache memory, other types of storage (e.g., EEPROM), and any suitable combination thereof. The term “machine-readable medium” should be taken to include a single medium or multiple media (e.g., a centralized or distributed database, or associated caches and servers) able to store processor instructions. The term “machine-readable medium” shall also be taken to include any medium, or combination of multiple media, which is capable of storing instructions for execution by one or more processors, such that the instructions, when executed by one or more processors cause the one or more processors to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” as used herein excludes signals per se.

What is claimed:

1. A method of monitoring a user's well-being, comprising:
creating an n-dimensional well-being profile specific to the user, the n-dimensional well-being profile comprising values for n well-being factors;
mapping at least one intervention activity to a plurality of well-being factors impacted by the intervention activity;
upon completion of the user performing an intervention activity, refining the n-dimensional profile into at least one n-dimensional sub-profile for an environment in which the intervention activity was performed, wherein the n-dimensional profile is refined in accordance with implicit and explicit responses to the completed intervention activity that impact the plurality of well-being factors in the environment;
providing a recommendation to the user for another intervention activity based on the refined at least one n-dimensional sub-profile for the environment; and
monitoring changes in at least one of the n-dimensional well-being profile or the at least one n-dimensional sub-profile to track changes in the user's well-being over time as the user completes intervention activities.

2. The method of claim 1, wherein the n well-being factors include factors that can directly or indirectly affect the user's well-being, including a plurality of cardio, resting heart rate, sleep hours, water intake, or smoking.

3. The method of claim 1, further comprising maintaining a library of the n well-being factors in a data store, the n well-being factors including at least one of calm, self-love, forgiveness, modesty, prudence, self-regulation, kindness, love, teamwork, leadership, diet, sleep, drugs, or community.

4. The method of claim 1, wherein the environment comprises one of work, home, a gym, or driving, and the user has an n-dimensional well-being profile for each environment.

5. The method of claim 1, further comprising assigning weights to well-being factors that are specific to the user and the user's environment to create the n-dimensional well-being profile, the weights being increased based on a number of implicit and explicit responses triggered by an associated well-being factor in response to an intervention activity.

6. The method of claim 1, further comprising generating trend and collaborative well-being features, the trend well-being features extrapolating intervention activities based on several users' completed intervention activities and recommending a specific intervention activity for the environment based on trends specific to the user, and the collaborative well-being features including well-being features from other users with a similar well-being profile to the user.

7. The method of claim 1, wherein the implicit and explicit responses relate to physiological and psychological effects of the completed intervention activity impacting the plurality of well-being factors in the environment, the implicit responses being measured by sensors and the explicit responses being provided by the user in response to a survey.

8. The method of claim 1, wherein mapping the at least one intervention activity to the plurality of well-being factors impacted by the intervention activity comprises a many-to-many mapping of the implicit and explicit responses to the completed intervention activity to the plurality of well-being factors impacted by the completed intervention activity.

9. The method of claim 1, further comprising computing an overall well-being profile for the user based on at least one n-dimensional sub-profile for each environment in which an intervention activity is performed by the user, the computing including summing respective well-being factors together for the at least one n-dimensional sub-profile for each environment.

10. The method of claim 1, further comprising collaboratively filtering the n-dimensional well-being profile specific to the user with an n-dimensional well-being profile specific to another user and providing the recommendation to the user for another intervention activity based on a result of the collaborative filtering.

11. The method of claim 1, further comprising storing the n-dimensional well-being profile specific to the user as versioned well-being factor values specific to the user and the environment.

12. The method of claim 11, wherein each time the well-being factors for a given n-dimensional well-being profile are updated, a new version of the n-dimensional well-being profile is created and a profile table is updated to track a version lineage of the n-dimensional well-being profile.

13. The method of claim 12, further comprising storing the n-dimensional well-being profile specific to the user in a hierarchical tree structure on a computing device where each new version of the n-dimensional well-being profile is stored under a current version of the n-dimensional well-being profile to create the at least one n-dimensional sub-profile.

14. The method of claim 13, wherein tracking changes in the user's well-being over time as the user completes intervention activities comprises tracking the n-dimensional well-being profile specific to the user in the hierarchical tree structure where each node in the tree represents a point in n-dimensional space.

15. The method of claim 14, further comprising identifying which well-being factors impact a user's well-being the most at each level in the hierarchical tree structure and recommending intervention activities for the identified well-being factors at each level in the hierarchical tree structure.

16. The method of claim 13, further comprising building the hierarchical tree structure for the user's n-dimensional well-being profile by:
obtaining a list of recommended well-being factors to pursue given the user's current position;
recommending intervention activities for each of the listed recommended well-being factors;
gathering feedback from the user to obtain an associated well-being label for the recommended well-being factors;
creating a new n-dimensional well-being profile for each intervention activity that uses a recommended well-being profile from a previous level in the hierarchical tree structure as its parent; and
selecting an n-dimensional well-being profile with a best well-being label as a candidate n-dimensional well-being profile for a current level in the hierarchical tree structure.

17. The method of claim 11, wherein storing the n-dimensional well-being profile specific to the user comprises storing the n-dimensional well-being profile specific to the user on a mobile computing device of the user, encrypting the n-dimensional well-being profile specific to the user into encrypted data, storing the encrypted data on a server, de-identifying the n-dimensional well-being profile specific to the user into de-identified aggregate profile data for a plurality of users, and storing the de-identified aggregate data on the server.

18. The method of claim 1, wherein refining the n-dimensional profile into at least one n-dimensional sub-profile for an environment in which the intervention activity was performed comprises incrementing values of the at least one n-dimensional sub-profile for the environment by a total number of implicit or explicit responses triggered by the completed intervention activity.

19. The method of claim 18, wherein an implicit response is triggered by the completed intervention activity when a value for a well-being factor affected by the completed intervention activity falls outside two standard deviations from historical values associated with the user for the well-being factor.

20. The method of claim 1, wherein refining the n-dimensional well-being profile into at least one n-dimensional sub-profile for an environment in which the intervention activity was performed comprises:
determining the user's environment;
measuring all implicit and explicit responses associated with well-being factors in the user's environment related to the recommended intervention activity;
determining which implicit responses impacted the well-being factors for the user in the user's environment by comparing a value of an implicit response against an historical value for the implicit response in that environment for the user;
when an explicit response is captured, determining when the explicit response was triggered;
for each implicit or explicit response that changed in value, determining the well-being factors that impact the implicit or explicit response;
for each well-being factor that impacts an implicit or explicit response, fetching records for an environment specific n-dimensional well-being profile specific to the user and determining from the records the values of well-being factors for the user for the environment specific n-dimensional well-being profile specific to the user;
incrementing a value of at least one well-being factor by a count of triggered implicit and explicit responses; and
updating the n-dimensional well-being profile with the value of the at least one well-being factor.

21. The method of claim 1, further comprising providing a well-being label that is used as a target variable to optimize while training a recommendation model for use in providing the recommendation to the user for another intervention activity based on the refined at least one n-dimensional sub-profile for the environment.

22. The method of claim 21, wherein training the recommendation model for another intervention activity occurs at a periodicity depending on how many intervention activities have been completed and the training uses variables extracted during the completed intervention activity as a feature vector and the well-being label as the target variable for the training.

23. The method of claim 21, wherein training the recommendation model for another intervention activity uses data extracted using a collaborative filter from other users' de-identified aggregate well-being profile data.

24. The method of claim 21, wherein training the recommendation model for another intervention activity includes predicting a sequence of well-being factors that is most likely to result in an improvement to the well-being label based on previous sequences of well-being factors that improved the well-being label in response to intervention activities mapped to the previous sequences of well-being activities.

25. A system for monitoring a user's well-being, comprising:
a hierarchical tree structure that stores an n-dimensional well-being profile specific to a user where each new version of the n-dimensional well-being profile is stored under a current version of the n-dimensional well-being profile to create at least one n-dimensional sub-profile comprising values for n well-being factors, respective well-being factors of the user being mapped to at least one intervention activity that impacts the respective well-being factors of the user;
a computing device that receives implicit and explicit responses to an intervention activity completed by the user that impacts the respective well-being factors of the user in an environment of the user, refines the n-dimensional well-being profile specific to the user into at least one n-dimensional sub-profile for the environment in which the intervention activity was performed, wherein the n-dimensional well-being profile is refined in accordance with the implicit and explicit responses to the completed intervention activity, and monitors changes in at least one of the n-dimensional well-being profile or the at least one n-dimensional sub-profile to track changes in the user's well-being over time as the user completes intervention activities; and
a personalized recommendation model that provides a recommendation to the user for another intervention activity based on the refined at least one n-dimensional sub-profile for the environment.

26. The system of claim 25, further comprising a server that receives and stores encrypted intervention activity completion data from the computing device and processes the intervention activity completion data to form a de-identified data stream.

27. The system of claim 26, wherein the server processes the de-identified data stream for a plurality of users to generate intervention activity recommendations for the user and pushes the intervention activity recommendations to the computing device.

28. The system of claim 25, further comprising a personalized training module that trains the personalized recommendation model using data extracted from the implicit and explicit responses to the completed intervention activity.
